(19) United States
(12) Patent Application Publication
(10) Pub. No.: US 2016/0015703 A1
(43) Pub. Date: Jan. 21, 2016

(54) NANO- MICRODELIVERY SYSTEMS FOR OROMUCOSAL DELIVERY OF AN ACTIVE INGREDIENT

(71) Applicant: Danmarks Tekniske Universitet, Kgs. Lyngby (DK)

(72) Inventors: Ioannis S. Chronakis, Lund (SE); Lars Jorgensen, Soborg (DK); Maria Ahlm Mattebjerg, Hvidovre (DK)

(21) Appl. No.: 14/772,462

(22) PCT Filed: Mar. 6, 2014
(86) PCT No.: PCT/EP2014/054331

§ 371 Date: Sep. 3, 2015

(30) Foreign Application Priority Data
Mar. 7, 2013 (EP) 13158191.0

(51) Int. Cl. A61K 31/465; A23L 1/22; A61K 9/00

(52) U.S. Cl. CPC A61K 31/465; A61K 9/006; A23L 1/22016; A23V 2002/00

(57) ABSTRACT

A composition for oromucosal delivery of at least one active ingredient, more particularly a lipid nano-microdelivery system comprising a nicotine component and/or a flavour component, wherein the nicotine component may be delivered to the oral cavity via absorption through the mucosal membranes thereof and/or wherein the flavour component may be delivered to the oral mucosa by controlled release.

### FIELD OF THE INVENTION

The present invention relates to a composition for oromucosal delivery of a nicotine and/or a flavour component. More particularly, the present invention relates to a lipid nano-microdelivery system comprising a nicotine and/or flavour component, wherein the nicotine component may be delivered to the oral cavity via absorption through the mucosal membranes thereof and/or wherein the flavour component may be delivered to the oral mucosa by controlled release.

### BACKGROUND OF THE INVENTION

Delivery of an active ingredient via the oromucosal route may be preferred in cases where delivery of said active ingredient via the saliva would present difficulties or disadvantages. Thus e.g. nicotine delivered via dissolution in the saliva often imparts a burning sensation and an unpleasant taste in the mouth. Thus a well-known side-effect of nicotine is related to its concentration dependent local irritation. This adverse effect is particularly noticeable when nicotine formulations are applied topically, including the transmucosal and transdermal administration routes. Moreover, an oromucosal delivery with a controlled and/or prolonged release of said active ingredient over time may be preferred, such as for flavour components.

WO 93/05768 relates to medication vehicles made of solid lipid particles with a diameter from 10 nm to 10 μm, that are solid at ambient temperature. Said medication vehicles are said to allow active substances to be controllably released over a longer period.

WO 99/15171 discloses nicotine compositions and methods of formulation thereof, said compositions comprising nicotine and, for reducing local nicotine-related irritation, a local analgesic or a mixture of local analgesics. WO 99/15171 also discloses a composition comprising nicotine, one or more polar lipids and one or more anionic surfactants in sufficient amounts to form a liquid crystalline phase or a precursor or offspring thereof when placed in a polar solvent.

WO 99/22703 discloses a formulation for topical application to a mucosal tissue, including the oral cavity, comprising a biologically active agent and a lipid carrier in the form of a colloidal composition which can include a micellar aggregate or mixed micelles dispersed in a continuous aqueous phase, wherein the lipid carrier includes at least one lipid selected from amphiphilic phospholipids, wherein said agent is carried by said lipid of said lipid carrier and said agent is released from said lipid in a sustained manner and over a prolonged period of time and wherein said lipid carrier has a property of high adhesion to the mucosal tissue.

WO 99/29301 relates to the use of a substantially non-aqueous composition comprising at least one membrane lipid or mono-acyl derivative thereof suspended in a hydrophilic medium in the manufacture of a composition for application to the mucosa as a soothing, protective or lubricating agent or as a carrier of a medicament in molecular dispersion.

WO 2007/113665 A2 discloses a polymerized solid lipid nanoparticle system comprising lipids and long chain fatty acids, a therapeutic protein or peptide, an adjuvant, a lectin, at least one polymer, and a pharmaceutically acceptable carrier. Administration of said system may be oral, sublingual or buccal.

WO 2010/104464 A1 relates to an oral delivery product comprising a semi-permeable pouch designed for being placeable in an oral cavity of a subject and multiple solid particles of at least one alginate salt of a monovalent cation and comprising at least one biologically active substance within a matrix formed by said at least one alginate salt of a monovalent cation, wherein said multiple solid particles are enclosed in said semi-permeable pouch. Said biologically active substance may be nicotine.

WO 2012/088059 A2 relates to a facile method for crosslinking and incorporating bioactive molecules into electrospun fiber scaffolds, wherein said scaffolds are crosslinked with an acrylate.

U.S. Pat. No. 4,880,634 relates to lipid nano-pellets as excipient system for perorally administered drugs, wherein the excipient system is in the form of an ultrafine aqueous, colloidal suspension of lipid nano-pellets comprised of lipids and a surfactant of which the particle diameters of the nano-pellets range from 50-1,000 nm.

U.S. Pat. No. 5,885,486 relates to solid lipid particles, particles of bioactive agents and methods for the manufacture and use thereof. More particularly the document relates to the preparation of suspensions of colloidal solid lipid particles of predominantly anisometrical shape with the lipid matrix being in a stable polymorphic modification and of suspensions of micron and submicron particles of bioactive agents.

US 2010/0247653 A1 discloses nanoparticles containing nicotine and/or cotinine, to dispersions containing nanoparticles and to transdermal pharmaceuticals containing nicotine and cotinine in nanoparticulate form.

Parhi et al., “Production of Solid Lipid Nanoparticles-Drug Loading and Release Mechanism”, J. Chem. Pharm. Res., 2010, 2(1): 211-227 reviews various production techniques for solid lipid nanoparticles.

There is a need in the art for nano-microstructures which can be utilised both in the solid state and in the dispersed state. More particularly, there is a need in the art for nano-microstructures which can be utilised without having to be dispersed/suspended in an aqueous phase. There is also a need for oromucosal delivery of nano-microsystems that do not disintegrate in saliva to release the active ingredient. There is also a need for nano-microsystems that adhere to the surface of the oral mucosa to release the active ingredient to the buccal mucosa.

There is also a need in the art for nano-microstructures that does not have a single diffusion behaviour of an active ingredient such as nicotine and/or flavour component but a controlled release of the active ingredient over time.

### OBJECT OF THE INVENTION

It is an object of embodiments of the invention to provide a composition for oromucosal delivery of an active ingredient so as to avoid saliva as dissolution medium for said active ingredient.

It is a further object of embodiments of the invention to provide a composition for oromucosal controlled delivery of an active ingredient.

It is a further object of embodiments of the invention to provide a composition providing a fast release of a first proportion of said nicotine and/or flavour component, preferably followed by a prolonged release of the remainder of said nicotine and/or flavour component.

### SUMMARY OF THE INVENTION

It has been found by the present inventor(s) that by providing a lipid nano-micro-structure incorporating a nicotine and/or flavour component therein it has surprisingly turned out that a desired oromucosal delivery of said nicotine and/or flavour component may be obtained.

So, in a first aspect the present invention relates to a composition for oromucosal delivery of at least one active ingredient comprising:

a lipid nano-micro-structure comprising at least one lipid and at least one active ingredient selected from the group consisting of a nicotine component and a flavour component, said at least one active ingredient being immobilised in said lipid nano-micro-structure.

In a second aspect the present invention relates to a method for the preparation of a composition in the form of lipid nano-microparticles according to the invention, comprising the steps of:
i) Providing said at least one lipid in a melted, optionally by heating to above the phase transition temperature thereof or providing said at least one lipid dispersed in a solvent;
ii) Dissolving said at least one active ingredient in the melted lipid, or in the lipid dispersed in a solvent;
iii) Optionally adding at least one surfactant, optionally at least one excipient and optionally at least one mucoadhesive compound and optionally at least one porogen compound neat or in a solution thereof, preferably an aqueous solution thereof;
iv) mixing and homogenising to obtain a nano-micro-structure comprising said at least one active ingredient component.

In a third aspect the present invention relates to a method for the preparation of a composition in the form of lipid nano-microfibres, comprising the steps of:
i) Providing said at least one lipid in a solvent, preferably an organic solvent or an organic solvent/aqueous system or in a supercritical fluid or using said at least one lipid in a melted state;
ii) Optionally adding at least one surfactant, optionally at least one excipient and optionally at least one mucoadhesive compound and optionally at least one porogen compound;
iii) Dispersing at least one active ingredient in the solution obtained in step ii);
iv) Applying an electrical field to obtain a composite nano-micro-structure.

In a fourth aspect the present invention relates to a use of the composition according to the invention for oromucosal delivery of said at least one active ingredient.

### LEGENDS TO THE FIGURE

FIG. 1 shows the release of nicotine from solid lipid nanoparticles (SLN),

FIG. 2 shows a Cryo-TEM image of SLN particles comprising nicotine,

FIG. 3 shows the size distribution of SLN particles comprising nicotine,

FIG. 4 shows an SEM image of R-Carvone electrospun lipid nanofibres,

FIG. 5 shows an SEM image of vanillin electrospun lipid nanofibres,

FIG. 6 shows the accumulated release of vanillin from electrospun lipid nanofibres, and

FIG. 7 shows the size distribution of SLN particles comprising R-carvone.

### DETAILED DISCLOSURE OF THE INVENTION

### Definitions

In the present context the term “oromucosal delivery” refers to delivery to the oral cavity. Examplary oromucosal delivery routes include i.a. buccal, sublingual and gingival delivery.

In the present context the term “solid lipid” is a lipid that is solid at room temperature and also at physiological body temperature.

In the present context the term “nano-micro structure” refers to a structure the size of which is in the nanometer and micrometer range, such as in the range 1 nm-1000 μm, such as 10 nm-1000 μm, such as 10 nm-100 μm, such as 10 nm-10 μm, such as 10 nm-1 μm, preferably in the range 1 nm-1 μm. The term also refers to a structure of any form, such as a sphere/beads, a fiber structure or any other shape wherein their average diameter is in the nanometer and micrometer range. The term also refers to a combination of fiber(s) and particles/beads; in particular where particle(s) structures are encapsulated/immobilised within fiber structures. This could e.g. be obtained using electrostatic processing methods (such as electrospray, electrocoextrusion and/or electrospinning, etc.).

In the present context the term “fiber”, “fibre”, and “fiber structure” refers to a structure having an oblong shape, i.e. wherein the length is at least 3 times the cross-section.

The term “solid lipid nano-microparticle” or “SLN-M” refers to solid lipid nano-microparticles having a solid lipid core matrix.

The term “nano-microstructured lipid carriers (N-MLC's)” is well-known in the art and disclosed in e.g. (R. H. Müller, M. Radtke, S. A. Wissing, ‘Solid lipid nanoparticles (SLN) and nanostructured lipid carriers (NLC) in cosmetic and dermatological preparations’, Advanced Drug Delivery Reviews, Volume 54, Supplement, 1 Nov. 2002, Pages S131-S155 as well in Nanoparticulates As Drug Carriers By V. P. Torchilin (editor) Imperial College Press, 2006, chapters 9 & 10). Nano-microstructured lipid carriers refer to structures that comprise a less organized solid lipid matrix, i.e. by blending a fluid lipid with the solid lipid. Generally, N-MLC's shows a higher loading capability for the immobilizing components than SLN-M.

The term “Lipid Drug Conjugate (LDC) Nano-microparticles” is well-known in the art and refers to nano-microparticles created through formation of insoluble lipid-drug conjugates either by salt formation or covalent linking like ester linkage. The formed LDC could be mixed into aqueous surfactant solution for preparation of SLN's by homogenization or other methods, cf. eg. Shaji J., Jain V. Solid Lipid Nanoparticles: A novel carrier for Chemoterapy, International Journal of Pharmacy and Pharmaceutical Sciences, Vol 2, Suppl 3, (1-10) 2010 and Olbrich C, Gebner A, Kayser O, Muller RH. Lipid-drug conjugate (LDC) nanoparticles as novel carrier system for the hydrophilic antitrypanosomal drug diminazene diaceturate. 3 Drug Target 2002; 10: 387-96.

The term “‘Polymer-Lipid hybrid Nano-microparticles” (PLNM) refers to nano-microparticles involving formation of complexation of compounds and an ionic polymer, cf. Shaji, supra; Wong H L, Bendayan R, Rauth A M, Wu X Y. Development of solid lipid nanoparticles containing ionically-complexed chemotherapeutic drugs and chemosensitizers. J Pharm Sci., 2004; 93: 1993-2004; Wong H L, Rauth A M, Bendayan R, Manias J L, Ramaswamy M, Liu Z et al. A new polymer-lipid hybrid nanoparticle system increases cytotoxicity of doxorubicin against multidrug resistant human breast cancer cells. Pharm Res 2006; 23: 1574-85; Wong H L, Bendayan R, Rauth A M, Xue H Y, Babakhanian K, Wu X Y. A mechanistic study of enhanced doxorubicin uptake and retention in multidrug resistant breast cancer cells using a polymer-lipid hybrid nanoparticle (PLN) system. J Pharmacol Exp Ther 2006; 317: 1372-81; and Li Y, Taulier N, Rauth A M, Wu X Y. Screening of lipid carriers and characterization of drug-polymer complex for the rational design of polymer-lipid hybrid nanoparticles. Pharm Res 2006; 23: 1877-87. Thus, charges on compounds are neutralized with polymer counter-ion and the formed complex is encapsulated into solid lipid nano-microparticles. Using this approach the encapsulation efficiency could be increased from typical 20-35% to over 80% and it is contemplated that the inclusion of ionic polymer in PLNM may accelerate the release rate of nicotine and/or flavour.

In the present context the term “diameter” of the nano-micro structure refers to the average diameter of the structure in question. Thus in connection with spheres the diameter is the average diameter of the spheres in question, and in connection with fibres the diameter is the average width of the fibres in question.

In the present context the terms “mucoadhesive” and “mucoadhesion” refers to the concept of a composition adhering to a mucous membrane. Mucoadhesive compounds facilitate mucoadhesion by their specific properties.

In the present context the term “excipient” refers to a compound which may be present in the composition according to the invention and which imparts desired properties thereto. An excipient may be used to regulate hydrophilicity and/or amphiphilicity and thereby control the release of the active ingredient from the nano-microstructure and may also be used as “carrier polymer”, e.g. as known in the electrostatic processing field.

In the present context the term “porogen” refers to a compound with pore-generating properties. The size of the porogen particles will affect the size of the pores in a polymer in question, while the polymer to porogen ratio is directly correlated to the amount of porosity of the final structure. Non-limiting examples of porogens are inorganic salts such as sodium chloride, and carbohydrate crystals, such as crystals of saccharose.

### Specific Embodiments of the Invention

In an embodiment of the invention said lipid nano-micro-structure is selected from the group comprising lipid nano-microparticles, lipid nano-microfibres, nano-microparticles encapsulated/immobilised in lipid nano-microfibres, lipid nano-microparticles encapsulated/immobilised in polymer nano-microfibers, lipid nano-microparticles encapsulated/immobilised in polymer nano-microparticles, and nano-microstructured lipid carriers (N-MLC's), and any combinations thereof, preferably wherein said lipid nano-micro-structure is selected from the group comprising solid lipid nano-microparticles and solid lipid nano-microfibres.

In an embodiment of the invention the active ingredient is a nicotine component.

In another embodiment of the invention the active ingredient is at least one flavour component.

In another embodiment of the invention the active ingredient is a combination of a nicotine component and at least one flavour component.

The amount of active ingredient may vary widely depending on the actual active ingredient used. However, typically an amount in the range 1-50% by weight of the lipid nano-microstructure is used, such as about 1-20% by weight.

Lipid nano-microstructures may take the form of either particles, fibres or nano-microstructured lipid carriers (N-MLC's) or a combination thereof, such as lipid nano-microparticles encapsulated/immobilised in nano-microfibres or lipid nano-microparticles encapsulated/immobilised in polymer nano-microparticles, or nano-microparticles encapsulated/immobilised in lipid nano-microfibres, lipid drug conjugate (LDC) nano-microparticles, polymer-lipid hybrid nano-microparticles (PLNM), or a combination of lipid nano-micro-structures wherein one of the active components (nicotine and/or flavor) is immobilised in separate lipid nano-micro-structures from the lipid nano-micro-structures immobilising the other component.

The lipid nano-micro-structure is preferably either solid lipid nano-microparticles or solid lipid nano-microfibres.

The solid lipid nano-micro-particles may be prepared by methods known in the art, e.g. as disclosed in Parhi et al, “Production of Solid Lipid Nanoparticles-Drug Loading and Release Mechanism”, J. Chem. Pharm. Res., 2010, 2(1):211-227. Solid lipid nano-microfibres may be prepared by electrospinning, e.g. as disclosed in McKee et al, “Phospholipid Nonwoven Electrospun Membranes” Science, 2006, 311: 353-355. Solid lipid nano-microbeads may be prepared by electrospraying or by using electrocoextrusion, e.g. as disclosed in N. J. Zuidam and E. Shimoni Chapter 2, Overview of Microencapsulates for Use in Food Products or Processes and Methods to Make Them, and N. J. Zuidam and V. A. Nedović (eds.), Encapsulation Technologies for Active Food Ingredients and Food Processing, DOI 10.1007/978-1-4419-1008-0 2, Springer Science & Business Media, LLC 2010.

Lipid nano-microparticles encapsulated/immobilised in nano-microfibres may be prepared using electrostatic processing methods such as electrospray, electrocoextrusion and/or electrospinning, etc. In one embodiment lipid nano-microparticles are added to a polymer solution and electrospun, electrosprayed or coelectroextruded together with the polymer solution into fibres or into other shapes, such as beads.

In an embodiment of the invention the lipid nano-micro-structures are in the form of Lipid Drug Conjugate (LDC) Nano-microparticles' produced through formation of insoluble lipid-(nicotine and/or flavour) conjugates either by salt formation or covalent linking like ester linkage. In an embodiment of the invention the formed LDC could be mixed into aqueous surfactant solution for preparation of SLN's by homogenization or other methods.

In another embodiment of the invention the lipid nano-micro-structures are in the form of Polymer-Lipid hybrid Nano-microparticles' (PLNM) which involves formation of complexation of nicotine and/or flavour and an ionic polymer. Charges on compounds such as nicotine and/or flavours are neutralized with polymer counter-ion and the formed complex is encapsulated into solid lipid nano-microparticles.

In an embodiment of the invention the nicotine and/or flavour component is present in the form of encapsulated material in the lipid nano-micro-structures.

In another embodiment of the invention the nicotine and/or flavour component is present in the form of a coating on lipid nano-micro-structures such as lipid nano-microfibres. This may be obtained e.g. by using coaxial electrospinning, or coaxial electrospray of colloidal suspensions, or electrocoextrusion of different liquids from coaxial capillaries.

In another embodiment of the invention the nicotine and/or flavour component is present in the form of a coating applied as a solution on the lipid nano-microparticles prepared as described above.

In an embodiment of the invention said nicotine component is selected from the group consisting of nicotine base and a salt of nicotine, such as nicotine bitartrate. The use of a salt of nicotine, such as nicotine bitartrate, may be beneficial in order to change the mechanism of absorption through the cell layers.

In an embodiment of the invention the composition comprises at least one flavour component. Although the range of flavours is almost limitless, flavours commonly fall into several broad categories, such as fruit flavours, spice flavours, and mint flavours. Flavours may be synthetic and natural or any combinations thereof. Non-limiting examples thereof include the following. Fruit flavours include lemon, orange, lime, grapefruit, tangerine, strawberry, apple, cherry, raspberry, blackberry, blueberry, banana, pineapple, cantaloupe, muskmelon, watermelon, grape, currant, mango, kiwi and mixtures thereof. Spice flavours include cinnamon, vanilla, clove, chocolate, nutmeg, coffee, liqorice, eucalyptus, ginger, lemongrass, cardamon, thyme, rosemary, anise and mixtures thereof. Mint flavours include spearmint, peppermint, wintergreen, basil, corn mint, menthol and mixtures thereof.

In an embodiment of the invention said flavour component is selected from the group consisting of one or more components of vanilla, such as vanillin, one or more components of spearmint such as R-carvone, one or more components of orange, such as limonene, menthol, one or more components of nutmeg, such as eugenol, of one or more components of eucalyptus, such as eucalyptol, one or more components of cinnamon, such as cinnamaldehyde, one or more components of thyme, such as thymol, one or more components of anise, such as anisole, one or more components of lemon or lime, such as citral.

In an embodiment of the invention said lipid nano-microstructure comprises at least one lipid selected from the group consisting of fatty acids, esters and fatty mono-, di-, and triglycerides thereof, partial glycerides, fatty alcohols and their esters and ethers, natural and synthetic waxes such as bees wax and carnauba wax, wax alcohols and their esters, hydrogenated vegetable oils, hard paraffins, phospholipids, sterols and sterol derivatives, and mixtures of any of the above lipids.

In an embodiment of the invention said at least one lipid is selected from the group consisting of C₈₋₂₄ fatty acids, C₈₋₂₄ fatty mono-, di-, or triglycerides, such as C₁₀₋₂₂ fatty acids, C₁₀₋₂₂ fatty mono-, di-, or triglycerides, such as saturated C₁₀₋₂₂ fatty acids and C₁₀₋₂₂ fatty mono-, di-, or triglycerides.

In an embodiment of the invention said at least one lipid is selected from the group consisting of capric, lauric, myristic, palmitic, stearic, and arachidic acids and mono-, di- and triglycerides thereof, preferably selected from trimyristin, tripalmitin, tristearin, tricaprin, myristic acid, palmitic acid, stearic acid, and behenic acid.

In an embodiment of the invention the lipid is a phospholipid preferably selected from the group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), PEOH (phosphatidyl alcohol), cholesterol, ergosterol and lanosterol, preferably phosphatidylcholine (PC).

In an embodiment of the invention the composition comprises at least one surfactant, wherein said at least one surfactant is selected from the group consisting of ionic, non-ionic, and amphoteric surfactants, preferably selected from the group consisting of non-ionic surfactants. Non-limiting examples thereof include polyvinyl alcohol (PVA), polyoxyethylene esters and ethers, such as Tween®80, SPAN®80 and Triton X-100, lecithin, sodium docecyl sulfate (SDS), copolymers of polyoxyethylene oxide and polyoxypropylene oxide, such as Poloxamer® 188, etc.

In an embodiment of the invention the at least one surfactant is present in a ratio of from about 1:0.005 to about 1:10 lipid nano-micro-structure:surfactant, preferably in the ratio of about 1:0.01 to about 1:0.1.

Thus the use of a surfactant(s) may stabilise the nano-micro-structure and prevent agglomeration of the individual nano-micro-particles. The use of a surfactant(s) may also control the morphology of the nano-microstructures that produced using electrostatic processing methods.

In an embodiment of the invention at least one porogen is present. Porogen leaching from the lipid nano-microstructures can be used to control the nano-microrpore size and porosity by selecting suitable amount of the added porogens, thus further controlling the release over time of the active ingredient. Modification of porogen surface to volume ratio can be used to optimize the permeability of the lipid nano-microstructure and thus control the release of the active ingredient.

In an embodiment of the invention the composition comprises a nicotine and a flavour component, wherein the nicotine component is immobilised in separate lipid nano-micro-structures from the lipid nano-micro-structures immobilising the flavour component. By incorporating the nicotine in separate lipid nano-micro-structures from the flavour component the release of both the nicotine component and the flavour component may be controlled to obtain e.g. a fast release of nicotine to satisfy the nicotine craving and a longer lasting flavour release to obtain a pleasant taste in the mouth.

In an embodiment of the invention the nicotine component is immobilised in solid lipid nano-micro-particles and the flavour component is immobilised in lipid nano-micro-fibres. Thereby it is possible to design the delivery of the nicotine and flavour to obtain the desired release profile.

In another embodiment of the invention the composition comprises a nicotine and a flavour component, wherein both components are present in the same lipid nano-micro-structures. Thereby it is possible to mask any undesired taste of the nicotine.

In another embodiment of the invention the composition comprises a nicotine and a flavour component, wherein a flavour component is encapsulated in the lipid nano-micro-structure and the nicotine is present as a coating on the surface thereof. Thereby a fast release of nicotine and a longer-lasting release of flavour can be obtained.

In an embodiment of the invention the composition further comprises at least one excipient, preferably wherein said excipient is a hydrophilic polymer and/or amphiphilic polymer, preferably selected from the group consisting of pectin, chitosan, dextran, pullulan, carrageenan, starch, cellulose acetate, sodium alginate, polyvinyl alcohol (PVA), polyacrylic acid (PAA), polyethylene oxide (PEO), methyl cellulose (MC), sodium carboxy methylcellulose (SCMC), hydroxy propyl cellulose (HPC), preferably selected from the group consisting of pectin, PEO, PVA and PAA. Such an excipient may further impart a desired hydrophilicity to the composition and may further control the dissolution rate and may thereby control the release of an active ingredient and to result in swelling and/or erosion of the lipid nano-microstructure.

In an embodiment of the invention the composition further comprises a compound having mucoadhesive properties in order to further control the delivery of the nicotine and/or flavour component to the desired place of delivery. A mucoadhesive compound may e.g. the included in the composition in the form of a coating of the lipid nano-micro-structure by polymer absorption or chemical crosslinking.

Non-limiting examples of a mucoadhesive compound include a compound selected from the group consisting of pectin, chitosan, sodium alginate, polyvinyl alcohol (PVA), polyacrylic acid (PAA), methyl cellulose (MC), sodium carboxy methylcellulose (SCMC), hydroxy propyl cellulose (HPC), preferably selected from the group consisting of pectin, PVA and PAA.

In an embodiment of the invention a composition in the form of lipid nano-microparticles or nano-micro lipid carriers (N-MLC's) is prepared by one of the following methods.

High Pressure Homogenization
a. Hot Homogenisation
   1. One or more lipids in a melted state are provided, optionally by heating to above the phase transition temperature thereof. Thus heating may not be necessary when the lipid(s) in question is/are in a liquid state at room temperature. However, in other embodiments heating will be applied in order to provide the lipid(s) in a melted state.
   2. the at least one active ingredient is dissolved or dispersed therein.
   3. any optional ingredients, such as surfactant(s), excipient(s) and/or mucoadhesive compound(s) and/or porogen(s) are added to the lipid dispersion either neat or as a solution, preferably an aqueous solution thereof, and mixed.
   4. thereafter the mixture obtained is subjected to high pressure homogenisation in a manner known per se, at an elevated temperature,
   5. thereafter the mixture obtained is cooled, to obtain the lipid nano-microparticles or N-MLC's.
b. Cold Homogenisation
   1. one or more lipids in a melted state are provided, optionally by heating to above the phase transition temperature thereof. Thus heating may not be necessary when the lipid in question is/are in a liquid state at room temperature. However, in other embodiments heating will be applied in order to provide the lipid(s) in a melted state
   2. the at least one active ingredient is dissolved or dispersed therein and rapidly cooled afterwards
   3. the above solid lipid mixture is milled to nano-micron size
   4. optionally, the above mixture is mixed with any optional ingredients, such as surfactant(s), excipient(s) and/or mucoadhesive compound(s) and/or porogen(s) that are added to the mixture either neat or as a solution, preferably as an aqueous solution thereof,
   5. thereafter the mixture obtained is subjected to high pressure homogenisation below the melting temperature of the lipid(s), to obtain the lipid nano-microparticles or N-MLC's.

Microemulsion Technique
1. one or more lipids in a melted state are provided, optionally by heating to above the phase transition temperature thereof. Thus heating may not be necessary when the lipid in question is in a liquid state at room temperature. However, in other embodiments heating will be applied in order to provide the lipid(s) in a melted state
2. the at least one active ingredient is dissolved or dispersed therein
3. separately, any optional ingredients, such as surfactant(s), excipient(s) and/or mucoadhesive compound(s) and/or porogen(s) in aqueous solution or dispersion are also heated to this temperature
4. the above two solutions or dispersions are then mixed together under mild stirring
5. the above hot mixture (pre-emulsion) is added to a cold water solution under mild stirring to obtain the lipid nano-microparticles or N-MLC's.

Solvent Emulsification-Evaporation Technique
1. one or more lipids and the at least one active ingredient are dissolved or dispersed in an water immiscible organic solvent,
2. any optional ingredients, such as surfactant(s), excipient(s) and/or mucoadhesive compound(s) and/or porogen(s) in aqueous solution or dispersion are added to the above mixture
3. the resulting solution or dispersion is emulsified using high speed homogenization
4. thereafter the organic solvent is removed (by e.g. rotary evaporator, etc.) to obtain the lipid nano-microparticles or N-MLC's.

Solvent Emulsification-Diffusion Technique
1. any optional ingredients, such as surfactant(s), excipient(s) and/or mucoadhesive compound(s) and/or porogen(s) in aqueous-solvent solution or dispersion of an aqueous-solvent is used, where the solvent is partially miscible with water,
2. one or more lipids and the at least one active ingredient are dissolved or dispersed in the above mixture, optionally with the use of heating
3. The above solution or dispersion is stirred to obtain an emulsion
4. The above emulsion is diluted with water to allow diffusion of the solvent to the aqueous phase
5. lipid nano-microparticles or N-MLC's are created by dilution of the above emulsion and
6. removal of the solvent (by e.g. rotary evaporator, etc.).

Melting Dispersion Method (Hot Melt Encapsulation Method)
1. one or more lipid(s) in a melted state are provided, optionally by heating to above the phase transition temperature thereof. Thus heating may not be necessary when the lipid in question is in a liquid state at room temperature. However, in other embodiments heating will be applied in order to provide the lipid(s) in a melted state
2. the at least one active ingredient is dissolved or dispersed therein; the above mixture then regarded as oil phase
3. simultaneously an aqueous phase containing any optional ingredients, such as surfactant(s), excipient(s) and/or mucoadhesive compound(s) and/or porogen(s) was also heated to same temperature as the oil phase
4. the above oil phase added in to a small volume of the aqueous phase and the resulting emulsion stirred at high speed stirring
5. the resulting emulsion is cooled to below the phase transition temperature thereof to obtain the lipid nano-microparticles or N-MLC's.

High Shear Homogenization and/or Ultrasonication Technique
1. one or more lipids and the at least one active ingredient are mixed in the melted state, optionally by heating to above the phase transition temperature of the lipid(s). Thus heating may not be necessary when the lipid(s) in question is/are in a liquid state at room temperature. However, in other embodiments heating will be applied in order to provide the lipid(s) in a melted state
2. any optional ingredients, such as surfactant(s), excipient(s) and/or mucoadhesive compound(s) and/or porogen(s) in aqueous solution or dispersion are heated and added to the above lipid-active ingredient mixture
3. the above solution or dispersion is emulsified in the melted state by probe sonication or using high speed stirring
4. the above solution or dispersion is cooled to below the phase transition temperature thereof to obtain the lipid nano-microparticles or N-MLC's.

Double Emulsion Technique
1. the at least one active ingredient is dissolved or dispersed with any optional ingredients, such as surfactant(s), excipient(s) and/or mucoadhesive compound(s) and/or porogen(s) in an aqueous solution or dispersion
2. the above solution or dispersion is emulsified with one or more lipid in the melted state, optionally by heating to above the phase transition temperature thereof. Thus heating may not be necessary when the lipid(s) in question is/are in a liquid state at room temperature. However, in other embodiments heating will be applied in order to provide the lipid(s) in a melted state
3. the above solution or dispersion mixture is then stirred and filtered to obtain the lipid nano-microparticles or N-MLC's.

Membrane Contactor Technique
1. one or more lipids and the at least one active ingredient are mixed in the melted state, optionally by heating to above the phase transition temperature of the lipid(s). Thus heating may not be necessary when the lipid(s) in question is/are in a liquid state at room temperature. However, in other embodiments heating will be applied in order to provide the lipid(s) in a melted state
2. the above mixture is pressed through membrane pores into a continuous flow of an aqueous solution or dispersion containing any optional ingredients, such as surfactant(s), excipient(s) and/or mucoadhesive compound(s) and/or porogen(s)
3. the above setup is kept above the lipid(s) melting temperature
4. afterwards, the outlet mixture is cooled below the lipid(s) melting temperature to obtain the lipid nano-microparticles or N-MLC's.
5.

Solvent Injection Technique
1. one or more lipid and the at least one active ingredient are dissolved or dispersed in an aqueous-miscible solvent or mixture thereof
2. the above solution or dispersion is pressed through a needle into a stirred aqueous phase, containing any optional ingredients, such as surfactant(s), excipient(s) and/or mucoadhesive compound(s) and/or porogen(s)
3. the above solution or dispersion is then filtered to remove excess of one or more lipids
   to obtain the lipid nano-microparticles or N-MLC's.

Supercritical Fluid Technology:

Gas/Supercritical/Antisolvent (GAS/SAS)
1. one or more lipids and the at least one active ingredient are dissolved or dispersed in an organic solvent with any optional ingredients, such as surfactant(s), excipient(s) and/or mucoadhesive compound(s) and/or porogen(s)
2. a supercritical fluid is dissolved in the above organic solvent mixture, causing liquid expansion
3. a precipitate is formed, due to the reduction of the ability of the solvent to dissolve the above mixture
4. lipid nano-microparticles or N-MLC's are formed thereby.

Gas Saturated Solution (PGSS)
1. one or more lipids and the at least one active ingredient are dissolved or dispersed in an solution with any optional ingredients, such as surfactant(s), excipient(s) and/or mucoadhesive compound(s) and/or porogen(s)
2. a supercritical fluid is dissolved at the above solution or dispersion mixture
3. the above solution or dispersion is subjected to a rapid depressurization, creating lipid nano-microparticles or N-MLC's.

In an embodiment of the invention a composition in the form of lipid nano-microfibres are prepared by providing at least one lipid in a solvent, preferably an organic solvent, an organic solvent/aqueous system or in a supercritical fluid. As organic solvents may be mentioned acetone, ccetic acid, 1,1,1,3,3,3-hexafluoro-2-propanol (HFIP), trifluoroacetic acid (TFA), trifluoroethanol, dichloromethane (DCM), chloroform, ethanol, formic acid,N,N-dimethyl formamide (DMF), hexafluoroacetone (HFA), among other and as supercritical fluids may be mentioned carbon dioxide and water. The choice of solvent depends on the electrostatic processing conditions specific for the composition, as known in the art. Any optional ingredients, such as surfactant(s), excipient(s), and mucoadhesive compound(s) and porogen(s) are added and the at least one active ingredient is dissolved or dispersed in the mixture obtained. Thereafter an electrical field is applied in a manner known per se to obtain a nano-microstructure in the form of nano-microfibres (or other shapes as known in the art).

In an embodiment of the invention a composition in the form of lipid nano-microfibres are prepared by providing at least one lipid in a melted state, optionally by heating to above the phase transition temperature thereof. Any optional ingredients, such as surfactant(s), excipient(s), and mucoadhesive compound(s) and porogen(s) are added and the at least one active ingredient is dissolved or dispersed in the mixture obtained. Thereafter an electrical field is applied in a manner known per se to obtain a nano-microstructure in the form of nano-microfibres (or other shapes as known in the art).

In an embodiment of the invention the composition is for use in chewing gums, lozenges, strips, orally dispersible powders, mouth sprays, pouches for oral use.

The composition according to the invention may be incorporated in any desired delivery form contemplated for oromucosal delivery.

The invention is disclosed in more detail below in the form of specific, non-limiting examples thereof.

### Example 1

### Preparation of Solid Lipid Nano-Particles (SLN) Comprising Nicotine

Solid lipid nano-particles comprising the following ingredients were prepared as disclosed below.

### Example 1a

Composition

Lipids:

Stearic acid 300 mg

Dynasan® 114, a microcrystalline triglyceride available from Sasol: 300 mg

Surfactants:

Polyvinylalcohol: 25 mg

Tween® 80: 25 mg

Active ingredient:

Nicotine base: 20 mg

### Example 1b

Composition

Lipids:

Stearic acid 400 mg

Hydrogenated sunflower oil: 200 mg

Surfactants:

Polyvinylalcohol: 25 mg

Tween®80: 25 mg

Active ingredient:

Nicotine base: 20 mg

The SLN's of examples 1a and 1b were prepared by melting the lipids to 80° C. and dissolving nicotine base therein. Tween® 80 was dissolved in 1 ml of water and the solution was mixed with the melted lipids by vortexing at 2000 rpm to obtain a crude pre-emulsion.

The mixed solution was transferred by syringe into a flask containing a solution of PVA in 50 ml of hot water at 85° C. The solution was subjected to homogenizing at 8000 rpm for 25 minutes to obtain solid lipid nanoparticles (SLN) containing nicotine.

The release of nicotine from the above SLN was tested in a buccal cell line (TR146) as shown in Table 1.

TABLE 1

| | Apparent permeability (cm/sec) |
|---|---|
| HBSS with nicotine* | 8.50E−06 |
| SLN with nicotine ** | 6.38E−06 |

*HBSS—Hank's Buffered Salt Solution with free nicotine
** SLP with nicotine in HBSS

As it appears the encapsulation of nicotine in SLN does not alter the kinetics of permeability since nicotine is released and transported across the cell layer to a similar degree.

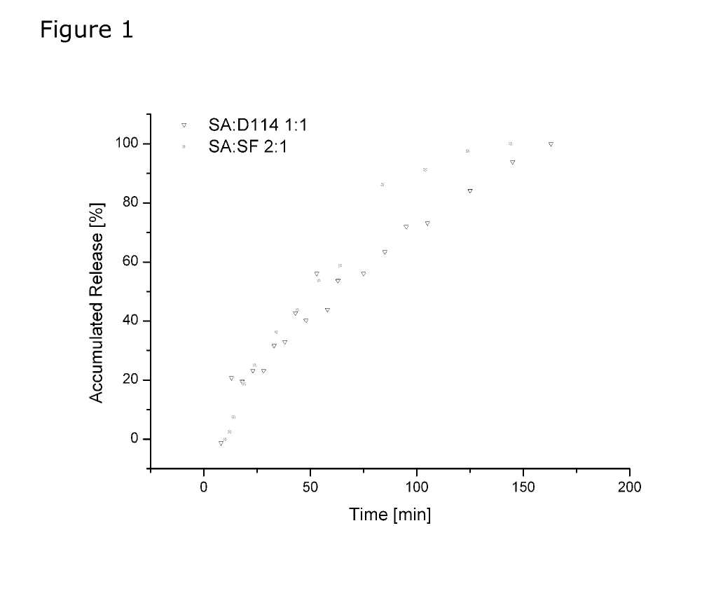

The release of nicotine over time was tested by placing the above SLN in a dialysis membrane placed in 200 ml of demineralised water and subjected to mechanical stirring at 300 rpm. The molecular weight cut-off of the dialysis membrane was 6-8 kDa. The result is shown in FIG. 1. “SA:D114” refers to Example 1a, and “SA:SF” refers to Example 1b. As it appears the full amount of nicotine is released in about 150 minutes.

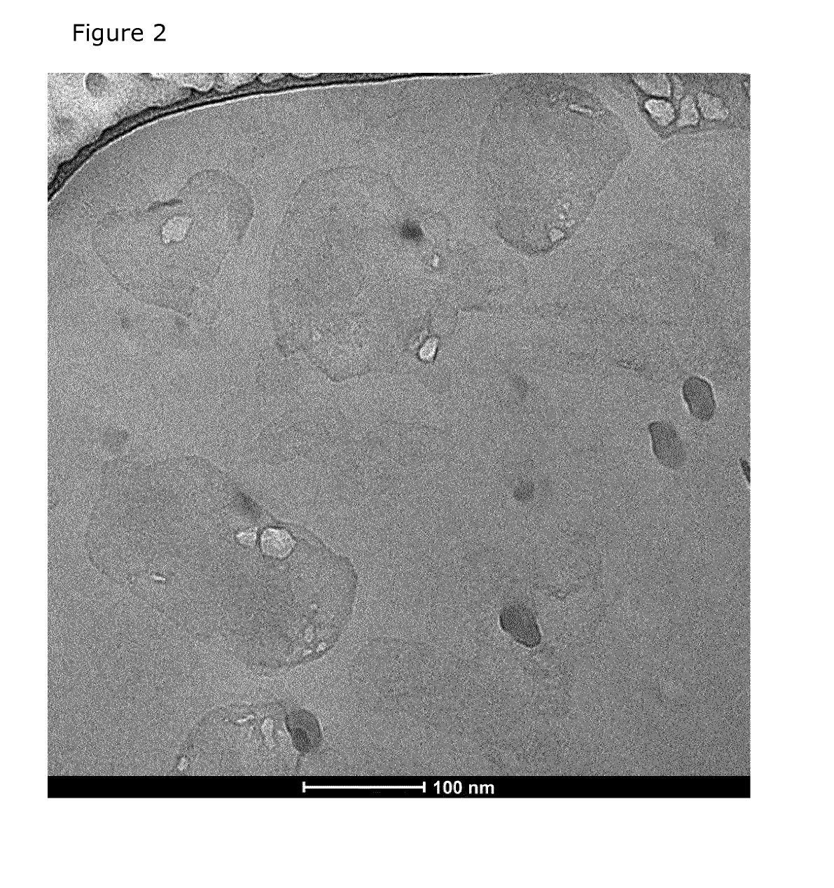

From FIG. 2 appears the formation of sphere-shaped SLN nanoparticles comprising nicotine, with sizes varies between 75 nm-200 nm

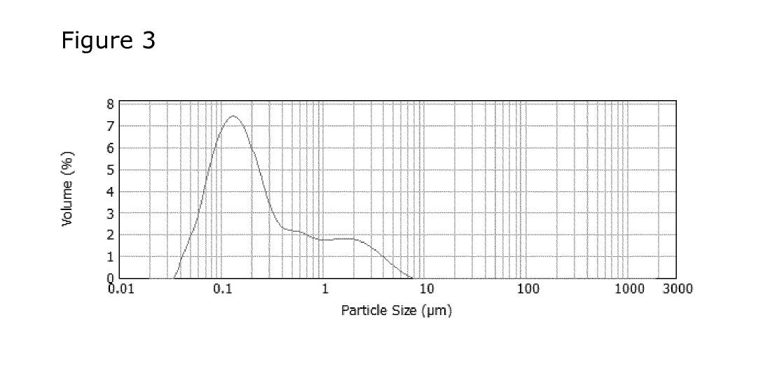

From FIG. 3 appears that the size distribution of SLN nanoparticles comprising nicotine, with a particle range between 50 nm to 10 μm and with main particle-size distribution between 50 nm to 250 nm.

### Example 2

### Preparation of Lipid Nanofibres Comprising R-Carvone

Lipid nanofibres were prepared by electrospinning.

A stock solution (SS1) of CHCl₃:DMF (2 ml CHCH3 and 1.33 ml DMF) was prepared.

1 g of lecithin was added to 0.96 ml of SS1 to give a 45% by weight lipid solution (LS). The solution was subjected to magnetic stirring for 30 minutes to dissolve the lipid. 30 mg of R-carvone was added. 10 μl of water was added to the lipid solution and the solution was left to stir overnight at low speed. Stirring was discontinued and the solution was left to stand for one hour before use.

The solution was transferred to a 5 ml syringe and subjected to spinning at a dispersion speed of 0.02 ml/min at a voltage of 27 kV and a distance of 6 cm.

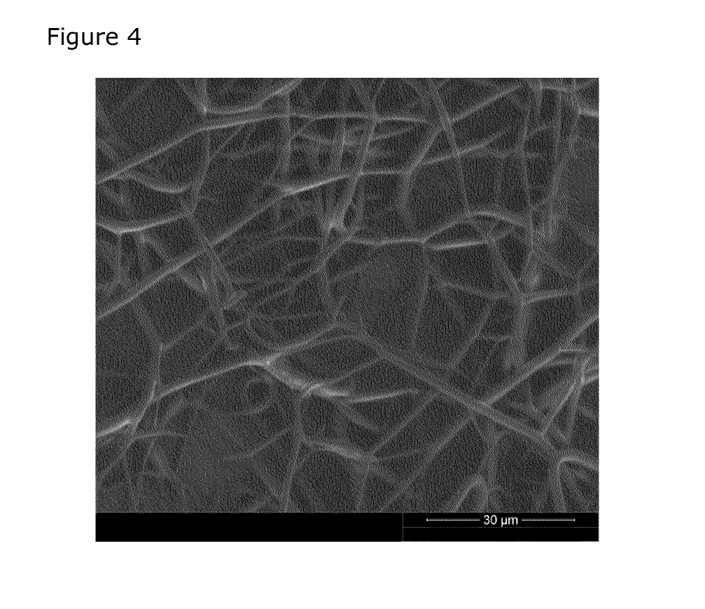

As it appears from FIG. 4 the flavor compound is fully encapsulated within the electrospun lipid nano-microstructures. The lipid nano-microfibers reveal a smooth surface without any formation of aggregates, indicating that the flavour was encapsulated homogeneously.

### Example 3

### Preparation of Lipid Nanofibres Comprising Vanillin

Lipid nanofibres comprising vanillin were prepared analogously to the procedure disclosed in Example 2 with the exception of a voltage of 28 kV and a distance of 10 cm being used and using 30 mg of vanillin instead of 30 mg of R-carvone.

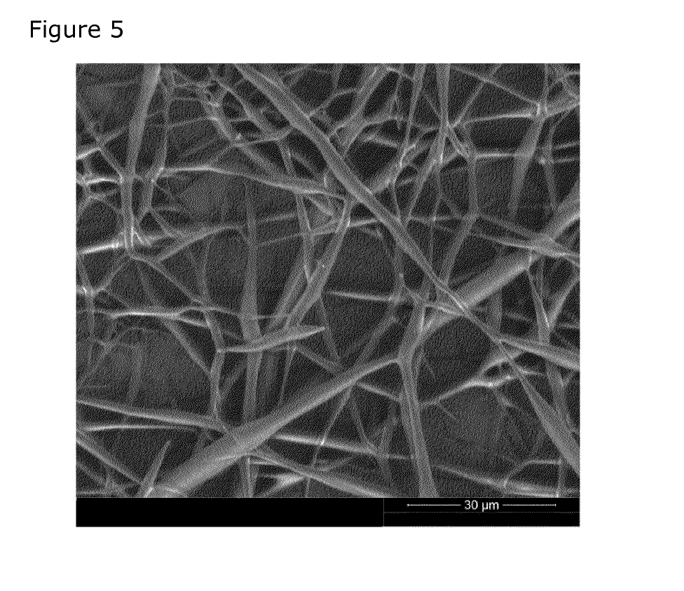

FIG. 5 shows an SEM image of electrospun lipid nano-microfibres with encapsulated vanillin. The lipid nano-microstructures showed homogeneous and smooth morphologies comparable to the morphologies as described at FIG. 4.

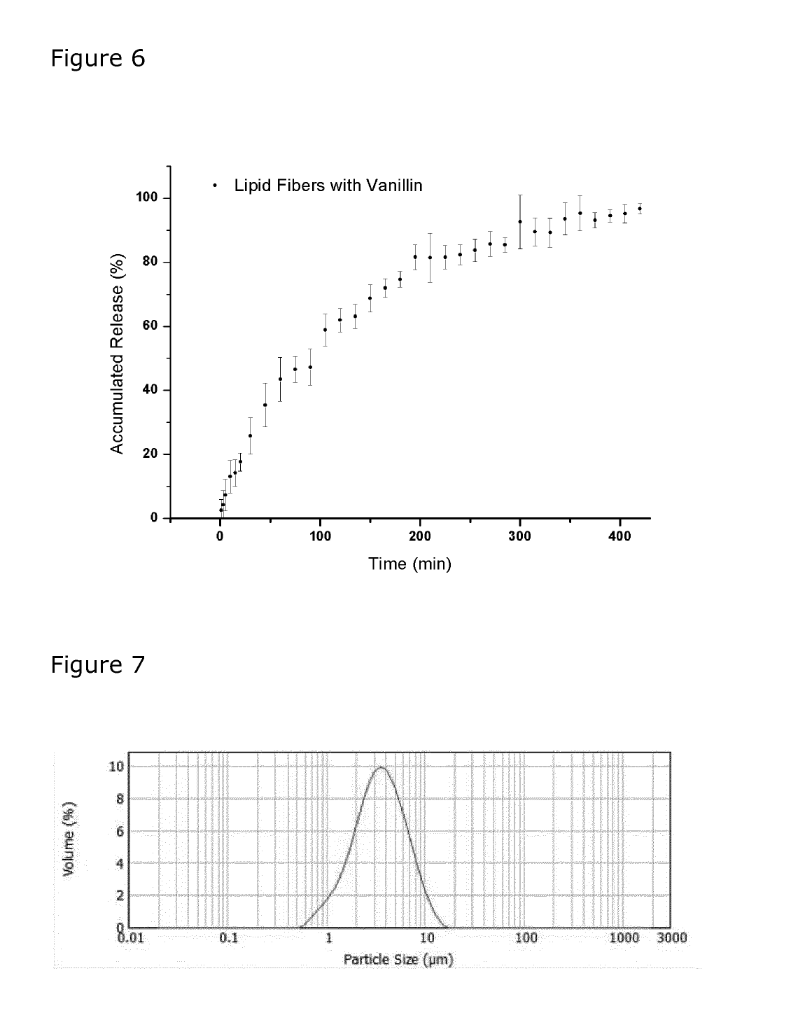

From FIG. 6 appears the accumulated release of vanillin from electrospun lipid nano-microfibres. As it appears the total amount of vanillin was released in about 400 minutes.

### Example 4

### Preparation of Solid Lipid Nanoparticles Comprising R-Carvone

Composition

Lipids:

Dynasan®114: 250 mg

Surfactants:

SPAN®80: 50 mg

Polyvinylalcohol: 15 mg

Tween® 80: 10 mg

Active ingredient:

R-(−)Carvone: 20 mg

The SLN of example 4 were prepared by dissolving the lipid in 500 μl Chloroform and dissolving R-(−)Carvone therein. Tween® 80 was dissolved in 1 ml of water and the solution was mixed with the dissolved lipids by vortexing at 2000 rpm to obtain a crude pre-emulsion.

The mixed solution was transferred by syringe into a flask containing a solution of PVA in 50 ml of cold water at 5° C. The solution was subjected to homogenizing at 8000 rpm for 60 minutes to obtain solid lipid nanoparticles (SLN) containing R-(−)Carvone.

### LIST OF REFERENCES

WO 99/15171
WO 99/22703
WO 99/29301
WO 2012/088059 A2
WO 2010/104464 A1
WO 2007/113665 A2
US 2010/0247653 A1
U.S. Pat. No. 4,880,634
U.S. Pat. No. 5,885,486
Parhi et al., “Production of Solid Lipid Nanoparticles-Drug Loading and Release Mechanism”, J. Chem. Pharm. Res., 2010, 2(1): 211-227

1. A composition for oromucosal delivery of at least one active ingredient comprising:
a lipid nano-micro-structure comprising at least one lipid and at least one active ingredient selected from the group consisting of a nicotine component and a flavour component, said at least one active ingredient being immobilised in said lipid nano-micro-structure.

2. The composition according to claim 1, wherein said lipid nano-micro-structure is selected from the group comprising lipid nano-microparticles, lipid nano-microfibres, nano-microparticles encapsulated/immobilised in lipid nano-microfibres, lipid nano-microparticles encapsulated/immobilised in polymer nano-microfibers, lipid nano-microparticles encapsulated/immobilised in polymer nano-microparticles, nano-micro structured lipid carriers (N-MLC's), lipid drug conjugate (LDC) nano-microparticles, and polymer-lipid hybrid nano-microparticles (PLNM), and any combinations thereof.

3. The composition according to claim 1, wherein said nicotine component is selected from the group consisting of nicotine base and a salt of nicotine.

4. The composition according to claim 1, wherein said flavour component is selected from the group consisting of one or more components of vanilla, one or more components of spearmint, one or more components of orange, menthol, one or more components of nutmeg, of one or more components of eucalyptus, one or more components of cinnamon, one or more components of thyme, one or more components of anise, and one or more components of lemon or lime.

5. The composition according to claim 1, wherein said lipid nano-microstructure comprises at least one lipid selected from the group consisting of fatty acids, fatty esters and fatty mono-, di-, and triglycerides thereof, partial glycerides, fatty alcohols and their esters and ethers, natural and synthetic waxes, bees wax, carnauba wax, wax alcohols and their esters, hydrogenated vegetable oils, hard paraffins, phospholipids, sterols and sterol derivatives, and mixtures thereof.

6. The composition according to claim 5, wherein said at least one lipid is selected from the group consisting of C8-24 fatty acids and C8-24 fatty mono-, di-, or triglycerides.

7. The composition according to claim 1 further comprising at least one surfactant, wherein said at least one surfactant is selected from the group consisting of ionic, non-ionic, and amphoteric surfactants.

8. The composition according to claim 7, wherein the at least one surfactant is present in a ratio of from about 1:0.005 to about 1:10 lipid nano-micro-structure:surfactant.

9. The composition according to claim 1, comprising a nicotine and a flavour component, wherein the nicotine component is immobilised in separate lipid nano-micro-structures from the lipid nano-micro-structures immobilising the flavour component.

10. The composition according to claim 1 further comprising at least one excipient.

11. The composition according to claim 1 further comprising at least one mucoadhesive compound selected from the group consisting of pectin, chitosan, sodium alginate, polyvinyl alcohol (PVA), polyacrylic acid (PAA), methyl cellulose (MC), sodium carboxy methylcellulose (SCMC), hydroxy propyl cellulose (HPC).

12. The composition according to claim 1, wherein said composition is selected from the group consisting of chewing gums, lozenges, strips, orally dispersible powders, mouth sprays, and pouches for oral use.

13. A method for the preparation of a composition in the form of lipid nano-microparticles according to claim 1, comprising the steps of:
a) Providing said at least one lipid in a melted state, optionally by heating to above the phase transition temperature thereof or providing said at least one lipid dispersed in a solvent;
b) Dissolving said at least one active ingredient in the melted lipid, or in the lipid dispersed in a solvent;
c) Optionally adding at least one surfactant, optionally at least one excipient and optionally at least one mucoadhesive compound and optionally at least one porogen compound neat or in a solution thereof; and
d) mixing and homogenising to obtain a nano-micro-structure comprising said at least one active ingredient component.

14. A method for the preparation of a composition in the form of lipid nano-microfibres according to claim 1, comprising the steps of:
a) Providing said at least one lipid in a solvent;
b) Optionally adding at least one surfactant, at least one excipient and optionally at least one mucoadhesive compound and optionally at least one porogen compound;
c) Dispersing at least one active ingredient in the mixture obtained in step b; and
d) Applying an electrical field to obtain a composite nano-micro-structure.

15. (canceled)

16. A method of oromucosal delivery of at least one active ingredient, said method comprising administering to a subject a composition of claim 1.

17. The composition according to claim 2, wherein said lipid nano-micro-structure is selected from the group comprising solid lipid nano-microparticles and solid lipid nano-microfibres.

18. The composition of claim 3, wherein said nicotine component is nicotine bitartrate.

19. The composition of claim 4, wherein said flavour component is selected from the group consisting of vanillin, R-carvone, limonene, eugenol, eucalyptol, cinnamaldehyde, thymol, anisole, and citral.

20. The composition according to claim 6, wherein said at least one lipid is selected from the group consisting of capric, lauric, myristic, palmitic, stearic, and arachidic acids and mono-, di- and triglycerides thereof.

21. The composition of claim 20, wherein said at least one lipid is selected from the group consisting of trimyristin, tripalmitin, tristearin, tricaprin, myristic acid, palmitic acid, stearic acid, and behenic acid.
